(19) United States
(12) Patent Application Publication
(10) Pub. No.: US 2004/0015095 A1
(43) Pub. Date: Jan. 22, 2004

(54) Method and device for measuring signals for electrical impedance tomography by using correlation techinique

(76) Inventors: Jianhua Li, Lubeck (DE); Steffen Leonhardt, Lubeck (DE)

Correspondence Address: McGLEW AND TUTTLE, P.C., SCARBOROUGH STATION, SCARBOROUGH, NY, 10510-0827, US

(21) Appl. No.: 10/406,522

(22) Filed: Apr. 3, 2003

(30) Foreign Application Priority Data
Jul. 16, 2002 (DE) 102 32 018.7

(51) Int. Cl. A61B 5/05

(52) U.S. Cl. 600/547

(57) ABSTRACT

A process for determining the correlation of signals of an electric impedance tomograph, in which one pair of electrodes each among a plurality of electrodes arranged in an annular pattern on the body is supplied with a harmonically time-dependent excitation current of frequency ω, and the measured voltage signals uᵢ(t) of the other passive electrode pairs are multiplied by a signal w(t)·sin(ω·t) or w(t)·cos(ω·t). ω is the frequency of the excitation current and w(t) is a window function, and they are subsequently integrated in order to obtain the real and imaginary parts of the signal uᵢ(t). To avoid a complicated multiplication by w(t)·sin(ω·t) or w(t)ωcos(ω·t) in a digital signal processor, provisions are made for a time interval of the signal w(t)·sin(ω·t) or w(t)·cos(ω·t) to be represented by a finite sequence of digital values and for sending these in phase with the harmonic excitation current of frequency ω to the digital input of a fast DA converter, while the signal uᵢ(t) to is sent to the reference voltage input of the DA converter, after which the analog signal sent from the DA converter is integrated and [sic—Tr.Ed.] the integrated analog signal is subjected to an AD conversion and is sent to a computing unit for further processing.

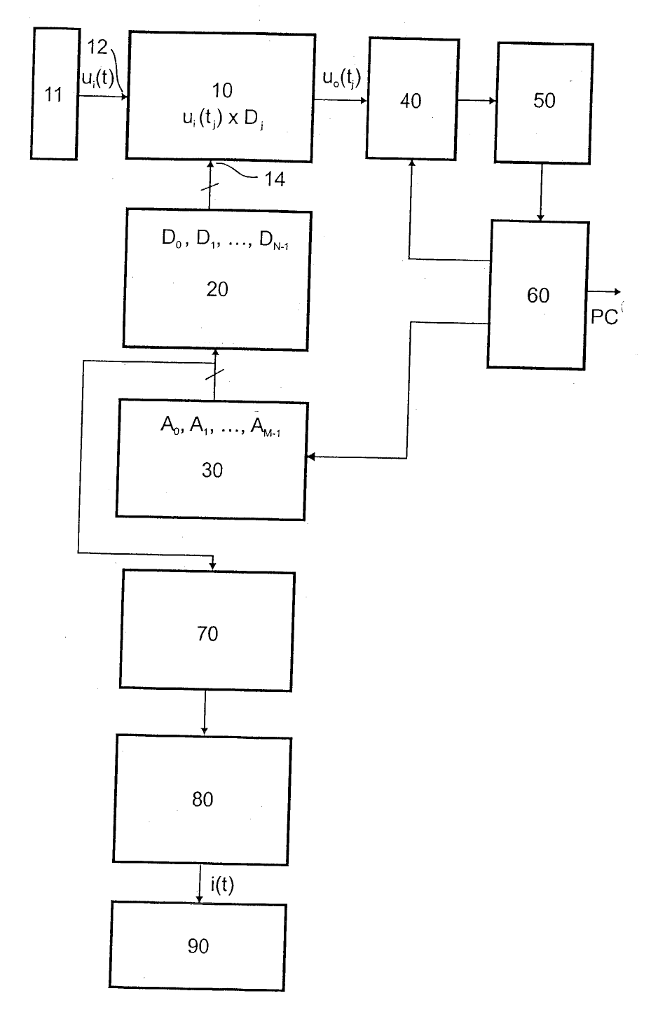

### FIELD OF THE INVENTION

The present invention pertains to a process and a device for measuring with correlation technique the signals of electrical impedance tomography, in which one pair of electrodes among a plurality of electrodes arranged in an annular pattern on the body is supplied with a harmonically time-dependent excitation current of frequency ω, and the measured voltage signals uᵢ(t) of the other passive electrode pairs are multiplied by a signal w(t)·sin(ω·t) or w(t)·cos(ω·t), wherein ω is the frequency of the excitation current and w(t) is a window function determining the duration of the measurement interval, and they are subsequently integrated in order to obtain the respective real and imaginary parts of the signal uᵢ(t).

### BACKGROUND OF THE INVENTION

Electric impedance tomography is an imaging method which is currently at the stage of practical development and shall be used especially for the regional analysis of pulmonary ventilation. A general description of the properties and the mode of operation of electric impedance tomography (EIT) can be found as a device for generating tomographic images in DE 43 32 257 C2. In electric impedance tomography (EIT), a plurality of electrodes, e.g., 16 electrodes, are arranged over the circumference of the chest in an annular pattern. For measurement, an electrode pair is first excited with an alternating current, and the voltage difference signals are measured at the remaining electrode pairs. All electrode pairs act consecutively as the feed electrode pair during one cycle, while the remaining other electrode pairs send voltage difference signals, which are then subjected to a further evaluation, and which finally yield a graphic image of the impedance distribution in the chest in a plurality of steps. The reconstruction algorithms, which yield two-dimensional impedance distributions from one or more measurement cycles, are not the subject of the present invention and will not be explained in greater detail here.

### SUMMARY OF THE INVENTION

The subject of the present invention is the correlation evaluation of the voltage difference signals uᵢ(t) of the passive electrode pairs, i.e., the signal processing at an early stage. Each signal uᵢ(t) of an electrode pair was hitherto multiplied for the evaluation of the correlation with a signal w(t)·sin(ω·t) or w(t)·cos(ω·t), wherein ω is the frequency of the excitation current, and the product was then integrated. The function w(t) is a “window function,” which shall determine a measurement interval. This signal processing was carried out hitherto such that the signal uᵢ(t) was fed into an AD converter with high sampling frequency and the digital values put out were subsequently multiplied by means of software by the signal w(t)·sin(ω·t) or w(t)·cos(ω·t) in a high-capacity digital signal processor. Such a procedure is possible within the framework of experimental studies and prototypes, but is far too expensive for a production apparatus, because AD converters with high sampling frequency and high-capacity digital signal processors are needed, whose cost is too high for practical use in a production apparatus for impedance tomography.

The object of the present invention is therefore to provide a process with which the correlation determination of the signals of the passive electrode pairs of an electric impedance tomograph can be accomplished with a simple design and at low cost.

The features of the invention are used to accomplish this object.

According to the invention a process for measuring the signals for an electric impedance tomography with a correlation technique is provided in which one pair of electrodes each among a plurality of electrodes arranged in an annular pattern on the body is supplied with a harmonically time-dependent excitation current of frequency ω. The measured voltage signals uᵢ(t) of the other passive electrode pairs are multiplied by a signal w(t)ωsin(ω·t) or w(t)·cos(ω·t), wherein to is the frequency of the excitation current and w(t) is a window function determining the duration of the measurement interval. They are subsequently integrated in order to obtain an indicator for the correlation. The signal w(t)·sin(ω·t) or w(t)·cos(ω·t) is represented in the time interval determined by the window function w(t) by a finite sequence of digital values, and these are sent to the digital input of a DA converter in phase with the harmonic excitation current, while the signals uᵢ(t) are sent to the reference voltage (or unit voltage) input of the DA converter, after which the analog signal sent from the DA converter is integrated, and the integrated analog signal is subjected to an AD conversion and is sent to a computing unit for further processing.

The sequence of digital values representing the signal w(t)·sin(ω·t) or w(t)·cos(ω·t) may be made available and stored in advance, and the sequence is later polled sequentially in phase of the harmonic excitation current and is sent to the digital input of the DA converter.

According to a further aspect of the invention, a DA converter, a memory, which contains the sequence of digital values (D₀, D₁, . . . , D_{N-1}) representing the signal w(t)·sin(ω·t) or w(t)·cos(ω·t), and an address generator my be provided. The address generator generates the addresses of the digital values in the memory as a function of the harmonic excitation current such that the sequence representing the signal w(t)·sin(ω·t) or w(t)·cos(•·t) is polled in phase with the excitation current of frequency ω and is sent to the digital input of the DA converter. An analog integrator and an AD converter may also be provided.

The memory may be a semiconductor memory, especially an EPROM or SDRAM memory.

The address generator may be a counter, which counts the number of values of the sequence representing the signal w(t)·sin(ω·t) or w(t)·cos(ω·t) once in phase with the excitation current during the measurement interval, each numerical value corresponding to one of the consecutive addresses of the values of the sequence in the memory.

Provisions are made according to the present invention for the function w(t)·sin(ω·t) or w(t)·cos(ω·t), with which the voltage signal uᵢ(t) must be multiplied, to be represented in the time interval defined by w(t) by a finite sequence of digital numbers. For illustration, this means that the continuous function is represented by a histogram or a step function with a finite number of digital amplitude values. The electrode pair voltage uᵢ(t) is sent to the reference voltage input of a DA converter. The sequence of digitized values of the signals w(t)·sin(ω·t) or w(t)·cos(ω·t) are sent to the digital input of the DA converter in phase with the excitation current. The output of the DA converter will then yield an analog signal, which corresponds to the product of the two signals and which is subsequently integrated in an analog integrator and is finally sent to an AD converter, from which it is sent to a digital data processing means for image reconstruction.

The correlation evaluation of the electric impedance tomograph can be carried out with little design effort with the process and the device according to the present invention.

The present invention will be described below on the basis of an exemplary embodiment based on a single figure, which shows a schematic block diagram for a device for carrying out the process. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which a preferred embodiment of the invention is illustrated.

### BRIEF DESCRIPTION OF THE DRAWING

In the drawing:

The only FIGURE is a schematic block diagram for a device for carrying out the process.

### DESCRIPTION OF THE PREFERRED EMBODIMENT

Referring to the drawing in particular, the block diagram in the figure shows a DA converter 10. The voltage signal uᵢ(t) of an electrode pair 11 is sent to the reference voltage (or unit voltage) terminal 12 of the DA converter 10.

A number of digital value D₀, D₁, . . . , D_{N-1}, which represent a discretized, digital representation of the function w(t)·sin(ω·t) or w(t)·cos(ω·t), are stored in an EPROM memory 20. Furthermore, there is an address generator 30, which may be designed as a counter and generates the addresses A₀, A₁, . . . A_{M-1} corresponding to the values D₀, D₁, . . . , D_{N-1} in the EPROM memory in a consecutive manner. Each digital value Dⱼ is sent to the digital input 14 of the DA converter 10. The addresses are generated over time such that the particular digitized values D₀, D₁, . . . , D_{N-1} of the function w(t)·sin(ω·t) or w(t)·cos(ω·t) is made available in phase with the excitation current of frequency ω.

As a result, the DA converter 10 sends an output voltage u₀(tⱼ) corresponding to the product uᵢ(tⱼ) at a time tⱼ. This output voltage is integrated in an analog integrator 40, whose output is finally sent to an AD converter 50, which is in turn connected to a microcontroller 60.

To ensure the synchronization of the excitation current and consequently of the signals uᵢ(t) with the supply of the discretized, digitized function D₀, D₁, . . . , D_{N-1}, i.e., to ensure that the sequence of digital values D₀, D₁, . . . , D_{N-1} representing the function w(t)·sin(ω·t) or w(t)·cos(ω·t) is in phase with the excitation current of frequency ω, provisions may be made, e.g., for the address generator 30 to also actuate an EPROM memory 70, in which a discretized, digitized sine or cosine signal is stored, and the digitized values are sequentially sent to a DA converter 80, from which the excitation current is obtained for the particular active electrode pair 90 of the impedance tomograph.

While a specific embodiment of the invention has been shown and described in detail to illustrate the application of the principles of the invention, it will be understood that the invention may be embodied otherwise without departing from such principles.

What is claimed is:

1. A process for measuring the signals for an electric impedance tomography with a correlation technique, the process comprising:
supplying one pair of electrodes among a plurality of electrodes arranged in an annular pattern on the body with a harmonically time-dependent excitation current of frequency ω;
multiplying the measured voltage signals uᵢ(t) of the other passive electrode pairs by a signal w(t)·sin(ω·t) or w(t)·cos(ω·t), wherein w is the frequency of the excitation current and w(t) is a window function determining the duration of the measurement interval, by representing the signal w(t)·sin(ω·t) or w(t)·cos(ω·t) in the time interval determined by the window function w(t) by a finite sequence of digital values, and these are sent to the digital input of a DA converter in phase with the harmonic excitation current, while the signals uᵢ(t) are sent to the reference voltage input of the DA converter;
subsequently integrating the signal sent from the DA converter in order to obtain an indicator for the correlation; and
subjecting the analog signal to an AD conversion and sending a resulting digital signal to a computing unit for further processing.

2. A process in accordance with claim 1, wherein the sequence of digital values representing the signal w(t)·sin(ω·t) or w(t)·cos(ω·t) is made available and stored in advance, and the sequence is later polled sequentially in phase of the harmonic excitation current and is sent to the digital input of the DA converter.

3. A device for measuring the signals for an electric impedance tomography with a correlation technique, the process comprising:
a device supplying one pair of electrodes among a plurality of electrodes arranged in an annular pattern on the body with a harmonically time-dependent excitation current of frequency ω;
a memory which contains the sequence of digital values (D₀, D₁, . . . , D_{N-1}) representing the signal w(t)·sin(ω·t) or w(t)·cos(ω·t);
an address generator generating the addresses of the digital values in the memory as a function of the harmonic excitation current such that the sequence representing the signal w(t)·sin(ω·t) or w(t)·cos(ω·t) is polled in phase with the excitation current of frequency ω;
a digital to analog (DA) converter multiplying the measured voltage signals uᵢ(t) of the other passive electrode pairs by the signal w(t)·sin(ω·t) or w(t)·cos(ω·t) polled in phase with the excitation current of frequency ω;
an analog integrator integrating the output of the DA converter; and
an analog to digital (AD) converter converting the integrated signal, wherein ω is the frequency of the excitation current and w(t) is a window function determining the duration of the measurement interval.

4. A device in accordance with claim 3, wherein the memory is a semiconductor memory.

5. A device in accordance with claim 4, wherein the memory is an EPROM or SDRAM memory.

6. A device in accordance with claim 3, wherein the address generator is a counter, which counts the number of values of the sequence representing the signal w(t)·sin(ω·t) or w(t)·cos(ω·t) once in phase with the excitation current during the measurement interval, each numerical value corresponding to one of the consecutive addresses of the values of the sequence in the memory.

7. A device in accordance with claim 4, wherein the address generator is a counter, which counts the number of values of the sequence representing the signal w(t)·sin(ω·t) or w(t)·cos(ω·t) once in phase with the excitation current during the measurement interval, each numerical value corresponding to one of the consecutive addresses of the values of the sequence in the memory.
